(19) United States
(12) Patent Application Publication
(10) Pub. No.: US 2023/0293882 A1
(43) Pub. Date: Sep. 21, 2023

(54) APPARATUS AND METHOD FOR TREATMENT OF NOCTURNAL ENURESIS

(71) Applicant: Adam Shattuck Howe, Albany, NY (US)

(72) Inventor: Adam Shattuck Howe, Albany, NY (US)

(21) Appl. No.: 18/184,225

(22) Filed: Mar. 15, 2023

Related U.S. Application Data
(60) Provisional application No. 63/320,204, filed on Mar. 15, 2022.

(51) Int. Cl. A61N 1/04; A61N 1/36

(52) U.S. Cl. CPC A61N 1/0456; A61N 1/36034; A61N 1/0476; A61N 1/0492

(57) ABSTRACT

Apparatus is provided for a TENS therapy apparatus and methods for use in treatment of nocturnal enuresis at home, easily, safely, and reliably. The apparatus and methods comprise a plurality of electrode pads with a range of novel and more effective pad placement locations, alone or in combination with an improved controller and treatment regimen. The apparatus and methods yield robust improvements in the frequency of enuresis and severity of wetness each night after TENS therapy, along with patient quality of life and convenience and ease of use for patient and caregivers.

### FIELD OF THE INVENTION

The presently disclosed subject matter relates to providing apparatus for treatment of nocturnal enuresis, and methods of use of the apparatus for treatment of nocturnal enuresis, and more particularly, to apparatus and methods of use of an apparatus for transcutaneous electrical nerve stimulation (TENS) for treatment of nocturnal enuresis.

### BACKGROUND OF THE INVENTION

Nocturnal enuresis is a common problem encountered in children, and causes additional upset and difficulty for children and their parents and other care-givers. Nocturnal enuresis can be recalcitrant to currently available treatment options. Primary monosymptomatic nocturnal enuresis in children may have several potential causes, including but not limited to those from the central or peripheral nervous systems. When initial behavioral management strategies (such as dietary, fluid, and bowel management modification) fail, nocturnal wetting alarms or pharmacotherapy is commonly initiated. Unfortunately, these efforts fail in a substantial percentage of patients.

Monosymptomatic nocturnal enuresis may have multiple underlying causes, and management strategies are limited. Primary nocturnal enuresis occurs in approximately 15% of five-year-old children with prevalence decreasing to 1-2% by age 15 years. Multiple management strategies have a range of shortcomings and failures. Behavioral and motivational therapy can lead to a significant improvement (>80% improvement in dry nights) in 70% of patients with a 5% relapse rate, but is expensive, time-consuming, may be slow to work, and is not highly likely to work. Bedwetting alarms retain nightly continence in 50% of patients. But, they require use for at least 3-4 months and have a 30% rate of discontinuation—again, highlighting that they are slow to work and not highly likely to work, providing children and their families disincentives to continue using them.

Pharmaceuticals can be used, such as desmopressin (the most commonly used pharmacological agent) which carries a 30% rate at achieving total dryness but also a 60-70% relapse rate after discontinuation. Imipramine has a 20% dry rate but a higher relapse rate (96%) after discontinuation. Pharmacological treatments can often come with other risks or side effects, and these treatments have a relatively high relapse rate, presenting another problem with their potential use.

Neuromodulation has not been studied extensively in children with refractory nocturnal enuresis, such as the foregoing. Neuromodulation techniques are known in the field, and are used to treat various urologic disorders. Transcutaneous electrical nerve stimulation (“TENS”) is a method that is known in the field, and can be used by individuals without medical training at home to treat nocturnal enuresis.

Prior art products exist, such as the “TheraPee” and other bedwetting alarm systems, and apparatuses that read biometric data from a patient and wakes them up when they need to void, such as the “GOGO Band”. Such examples contain the same shortcomings as described above. Another method for treatment of nocturnal enuresis is posterior tibial nerve stimulation (“PTSN”), which is a needle placed into the posterior tibial nerve. This is a therapy that is only used in a urology clinic or similar, and is not available for treatment at home. There are other clinical or surgical techniques used for treatment of nocturnal enuresis, such as a surgically implanted apparatus for sacral neuromodulation, that is, sacral nerve stimulation. This is a therapy that is only available through surgery, with all of the concomitant risks of surgery and the expense, and inconvenience.

TENS is a less invasive and more available form of neuromodulation that is FDA-approved and has been used in various conditions including for the treatment of urinary symptoms. Rare adverse reactions such as pain and skin irritation are managed with proper hygiene, decreasing the intensity setting, and periodic removal of the pads. In children with lower urinary tract symptoms, TENS units placed sacrally have shown promising results with multiple sessions weekly, and other studies showed posterior tibial nerve stimulation using a TENS unit improved daytime and nighttime voiding disorders.

TENS therapy has been used to attempt to treat a variety of pediatric voiding conditions. The successful use of neuromodulation to treat symptoms of overactive bladder, urinary retention, and fecal incontinence in children refractory to pharmacologic therapy, that is, for whom medications have not worked, is well established. Neuromodulation has generally been employed as either a surgically implanted device placed in two stages, with electrodes positioned through the S3 foramen, or in the form of posterior tibial nerve stimulation, with electrodes placed percutaneously during weekly sessions in an office setting. For these conditions, treatment has typically used a parasacral location of the TENS stimulation apparatus. In children, parasacral neuromodulation has been thought to be more effective than posterior tibial nerve stimulation in resolving symptoms of overactive bladder, but parasacral neuromodulation has yielded similar rates of resolution of incontinence. A significant shortcoming with TENS therapy to date has been low efficacy for treatment of nocturnal enuresis. Lordêlo et al. evaluated TENS units on nonmonosymptomatic enuresis patients placed parasacrally and found improvement or resolution in nocturnal episodes in most patients, but unfortunately 30-40% failed to reach complete resolution (Lordêlo P, Benevides I, Kerner E G, Teles A, Lordêlo M, Barroso U Jr. Treatment of non-monosymptomatic nocturnal enuresis by transcutaneous parasacral electrical nerve stimulation. J Pediatr. Urol. 2010 October; 6(5):486-9). Oliveira et al. used this same protocol for monosymptomatic enuresis and, despite a reduction in wet nights, no patient was cured of enuresis (Oliveira L F, Oliveira D M, Silva de Paula L I, Figueiredo A A, Bessa J Jr, Sá C A, Bastos Netto J M. Transcutaneous parasacral electrical neural stimulation in children with primary monosymptomatic enuresis: a prospective randomized clinical trial. J Urol. 2013 October; 190(4):1359-63). A further shortcoming of parasacral placement is that placing the pads in the proper location at home without a provider can be challenging, and the patient requires another person to place these pads which can be embarrassing to older patients, including older children.

A further shortcoming with the methods and apparatus known in the art for TENS is that the protocols in the literature are performed in the clinic setting. This can be inconvenient for the children and their care-givers, and thus can negatively affect compliance and use of the methods and apparatuses—making them less useful and less appealing. In 2017, Ferroni et al. studied the effects of nightly TENS with electrode pads placed on the foot for two weeks and saw a slight improvement in dry nights with sustained improvement two weeks after treatment without adverse reactions (Ferroni M C, Chaudhry R, Shen R, et al. Transcutaneous Electrical Nerve Stimulation of the Foot: Results of a Novel At-home, Noninvasive Treatment for Nocturnal Enuresis in Children. Urology. 2017 March; 101:80-84).

Other treatment options that have been studied and used for nocturnal enuresis include electro-acupuncture. Electro-acupuncture has been successful with long duration of treatment, but multiple treatment sessions were required, presenting a shortcoming with this treatment option.

In the art, others have attempted to treat nocturnal enuresis by activating or using the pathways in the region of the ankle (posterior tibial) location. Combining behavioral therapy with pelvic floor muscle training, biofeedback, has had marginal success, and intra-anal electric muscle stimulation demonstrated a delayed but improved partial response in 86.7% and complete response in 13.3% three months after treatment. Shortcomings include the time required and the expense and difficulty of treating children with these methods.

Acupuncture, which is based off the main meridian channels of the body, has been shown to be effective in treating nocturnal enuresis. Shortcomings include the time required and the expense and difficulty of treating children with these methods, and the fact that it is not a treatment method that can be applied at home.

The prior art does not meet the needs for a treatment for nocturnal enuresis that is safe, effective, and can be used at home by anyone, specifically by non-medical professionals.

### SUMMARY OF THE INVENTION

The present invention meets all these needs, by disclosing an apparatus for treatment of nocturnal enuresis and methods for use of the apparatus for such treatment. The present invention addresses the problems of the prior art, which do not present an apparatus or methods that meets all of these needs, by being safe, effective, and simple to use at home by anyone.

The present disclosure presents a TENS apparatus with controls and settings optimized for treatment of nocturnal enuresis, and methods for using the TENS apparatus for treatment of nocturnal enuresis that are simplified and optimized for use by young patients and their caregivers. With the apparatus, the posterior tibial (ankle) location is the most favorable for TENS therapy, in contradistinction to the parasacral location which has been used more commonly in prior TENS studies for other pediatric voiding conditions. The posterior tibial location offers improvements in efficacy and comfort. The posterior tibial nerve lies along the inferior portion of the kidney channel, and the electrode pad of the present disclosure is placed right at the KI-3 acupoint which is used in acupuncture and electroacupuncture protocols for treating enuresis. Other common acupoints for treating voiding symptoms along the posterior tibial nerve are KI-7, KI-10, SP-3, and SP-6. The CV-2-4 points are commonly used and are located in the same area as our suprapubic group, which also had good results in our study. BL-23 and BL-28 are used as well and located in the parasacral region. Our new discoveries with TENS therapy improve on acupuncture treatments for patients, and improving our knowledge of acupuncture will help drive further developments in novel treatments for nocturnal enuresis. While it is true that the location of the posterior tibial nerve has been targeted in other studies, such as a foot reflexology study that was found to completely eliminate nocturnal enuresis in an 8-year-old child with attention-deficit hyperactivity disorder, reflexology is not a simple or at-home treatment that is accessible to children and their caregivers without disruption to their lives. In addition, a study of weekly percutaneous posterior tibial nerve stimulation with a good treatment response in refractory monosymptomatic nocturnal enuresis patients after 12 weeks, but this invasive approach is not practical for children and not simple or accessible for a large range of patients. In the present disclosure, the electrode pads can be placed easily by the patient or the patient's caregiver, and the electrical impulses can penetrate the posterior tibial nerve easily due to the thinner skin in this area of the body, as compared to the pad placement locations that have been previously known and used in the art of treatment of nocturnal enuresis.

The apparatus and methods presented have led to significantly improved quality of life during TENS therapy, along with robust improvements in the frequency of enuresis and severity of wetness each night after TENS therapy. The posterior tibial (ankle) location described herein has been found to be the most favorable for TENS therapy, in contradistinction to the parasacral location which has been used more commonly in the art in using TENS for other pediatric voiding conditions. TENS therapy when combined with behavioral techniques can be an easy and safe tool that can be used reliably at home for treating nocturnal enuresis.

The present disclosure examines multiple parameters showing improved quality of life, frequency and severity of nocturnal enuresis, durability, excellent compliance, patient satisfaction, and safety with ankle TENS therapy without any adverse reactions or side effects. The apparatus and methods of the present disclosure were compared head-to-head with multiple different TENS locations in treating nocturnal enuresis. The present disclosure presents a protocol for a safe and easy-to-use home therapy that can be used before bed, which is convenient to families, and without medications.

In one aspect of the present disclosure, the disclosure comprises an apparatus for treatment of nocturnal enuresis, the apparatus comprising a control unit, a controller, wherein the controller is in electrical or mechanical communication with the control unit, a band, a plurality of electrical leads, and a plurality of electrode pads; wherein the control unit generates a plurality of electrical impulses for transcutaneous electrical nerve stimulation (TENS).

In one aspect of the present disclosure, the apparatus comprises the controller is adjustable to control an intensity of the plurality of electrical impulses.

In one aspect of the present disclosure, the apparatus comprises the band is reversibly affixed to the control unit, and the band reversibly affixes the apparatus to an ankle, a foot, a leg, or another body part of a patient.

In one aspect of the present disclosure, the apparatus comprises the plurality of electrical leads are in electrical communication with the control unit and with the plurality of electrode pads, such that the plurality of electrical impulses may be transmitted from the control unit through the electrical leads to the plurality of electrode pads, where the plurality of electrical impulses can penetrate a plurality of skin of a patient.

In one aspect of the present disclosure, the apparatus comprises the plurality of electrode pads are used in a plurality of pad placement locations.

In one aspect of the present disclosure, the apparatus comprises the plurality of electrode pads are used in an ankle (posterior tibial) pad location, and wherein the ankle (posterior tibial) pad location comprises a location for one or more of the plurality of electrode pads on an ankle and a location for one or more of the plurality of electrode pads on a foot, referred to as a foot pad location.

In one aspect of the present disclosure, the apparatus comprises the ankle (posterior tibial) pad location, to create an indirect distal neural loop, means the plurality of electrode pads have a plurality of pad placement locations on a bottom of a foot of a patient, and over a posterior portion of a medial malleolus on the ankle that is ipsilateral thereto.

In one aspect of the present disclosure, the apparatus comprises the control unit omits a control for waveform of the plurality of electrical impulses.

In one aspect of the present disclosure, the apparatus comprises the control unit is set to a pulse rate of approximately 10 Hz, normal mode, with a waveform pulse that is rectangular with a width of approximately 270 us, a therapy-duration time set to 15 min, and a pulse intensity that is determined either programmatically, or by a medical provider based on a tolerance threshold of a patient, or by the patient and/or a caregiver.

In one aspect of the present disclosure, the apparatus comprises the control unit further comprises an activation control and an intensity control.

In one aspect of the present disclosure, the disclosure comprises a method for transcutaneous electrical nerve stimulation treatment (TENS treatment) of nocturnal enuresis, the method using an apparatus of claim 1, wherein the method comprises, for a patient, reversibly affixing the apparatus to the patient, at least nightly; and performing TENS therapy at a therapy-frequency for a therapy-span; and wherein each time the TENS therapy is conducted, the TENS therapy is conducted for a therapy-duration.

In one aspect of the present disclosure, the method comprises the method further comprises treating the patient with behavioral therapy for a duration of time prior to the TENS treatment.

In one aspect of the present disclosure, the method comprises the duration of time prior to the TENS treatment is approximately one month.

In one aspect of the present disclosure, the method comprises the method further comprises having the patient keep a voiding diary.

In one aspect of the present disclosure, the method comprises the therapy-span is approximately a month.

In one aspect of the present disclosure, the method comprises the therapy-frequency is daily.

In one aspect of the present disclosure, the method comprises the therapy-duration is approximately 15 minutes.

In one aspect of the present disclosure, the method comprises a control unit of the apparatus is set to a pulse rate of approximately 10 Hz, normal mode, with a waveform pulse that is rectangular with a width of approximately 270 us, a therapy-duration time set to 15 min, and a pulse intensity determined either programmatically, or by a medical provider based on a tolerance threshold of the patient, or by the patient and/or a caregiver.

These aspects of the present invention, and others disclosed in the Detailed Description of the Drawings, represent improvements on the current art. This summary is provided to introduce a selection of concepts in a simplified form that are further described below in the Detailed Description of the Drawings. This Summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of various aspects, is better understood when read in conjunction with the appended drawings. For the purposes of illustration, the drawings show exemplary aspects; but the presently disclosed subject matter is not limited to the specific methods and instrumentalities disclosed. In the drawings, like reference characters generally refer to the same components or steps of the device throughout the different figures. In the following detailed description, various aspects of the present invention are described with reference to the following drawings, in which:

FIG. 1 shows a lateral view of a person's foot with the apparatus in place, in an aspect of the apparatus of the present invention.

FIG. 2 shows a plantar and posterior view of a person's foot with the apparatus in place, in an aspect of the apparatus of the present invention.

FIG. 3 shows a medial view of a person's foot with the apparatus in place, in an aspect of the apparatus of the present invention.

FIG. 4 shows a dorsal and anterior view of a person's foot with the apparatus in place, in an aspect of the apparatus of the present invention.

FIG. 5 shows a schematic view of a method of use of an aspect of the apparatus of the present invention.

FIG. 6 shows a Consolidated Standards of Reporting Trials (CONSORT) chart of the design of a study testing the apparatus and method of use of the present disclosure.

FIG. 7 presents a chart of percentage improvement of wet night frequency under the study conducted with the apparatus and method.

FIG. 8 presents a chart of improvement in wet scale severity scores under the study conducted with the apparatus and method.

FIG. 9 presents a chart of improvement in PIN-Q scores under the study conducted with the apparatus and method.

### DETAILED DESCRIPTION OF THE DRAWINGS

The presently disclosed invention is described with specificity to meet statutory requirements. But, the description itself is not intended to limit the scope of this patent. Rather, the claimed invention might also be configured in other ways, to include different steps or elements similar to the ones described in this document, in conjunction with other present or future technologies. Moreover, although the term “step” or similar terms may be used herein to connote different aspects of methods employed, the term should not be interpreted as implying any particular order among or between various steps herein disclosed unless and except when the order of individual steps is explicitly described. The word “approximately” as used herein means within 15% of a stated value, and for ranges as given, applies to both the start and end of the range of values given.

In the following description, numerous specific details are set forth to provide a thorough understanding of the invention. But, the present invention may be practiced without these specific details. Structures and techniques that would be known to one of ordinary skill in the art have not been shown in detail, in order not to obscure the invention. Referring to the figures, it is possible to see the various major elements constituting the apparatus and methods of use the present invention.

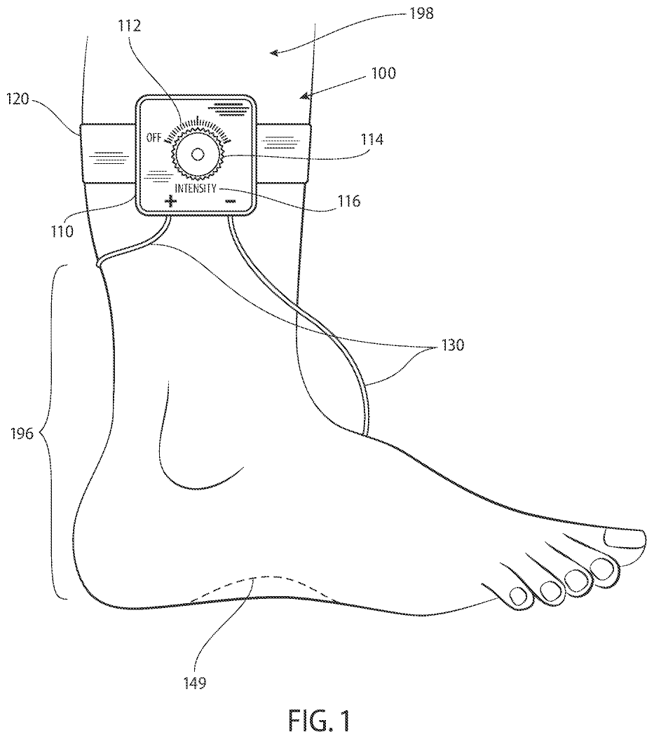

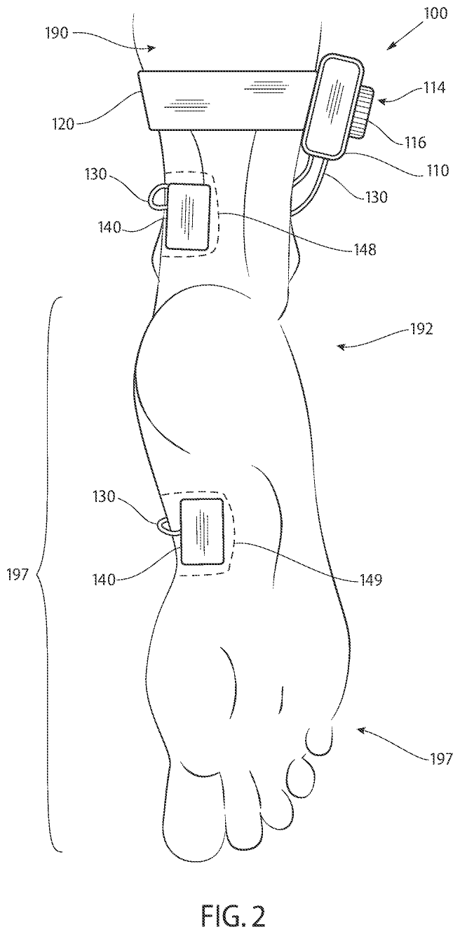

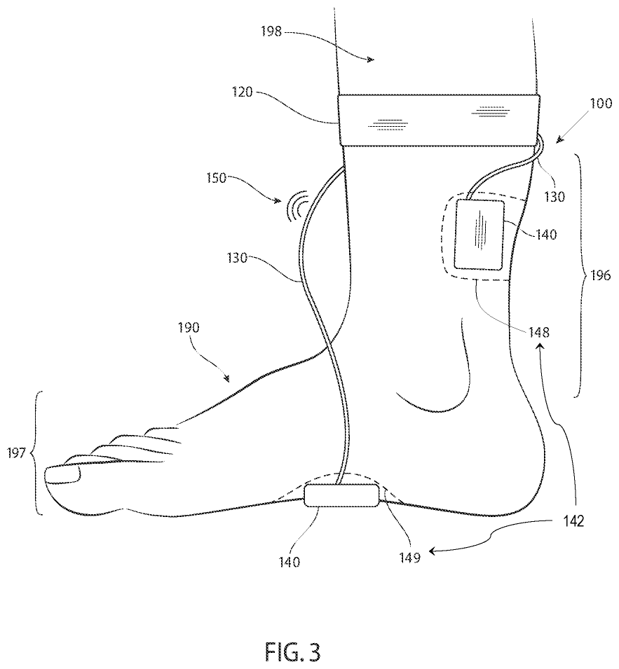

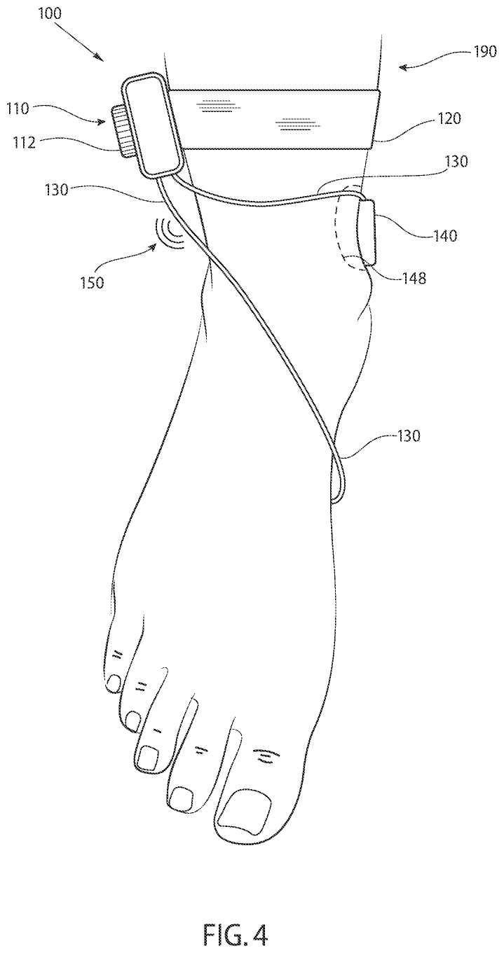

With reference to FIG. 1, FIG. 2, FIG. 3, and FIG. 4, the present invention comprises an apparatus 100 for TENS for treatment of nocturnal enuresis. The apparatus 100 comprises a control unit 110, a controller 112 in electrical or mechanical communication with the control unit 110, a band 120, a plurality of electrical leads 130, and a plurality of electrode pads 140. The control unit 110 generates a plurality of electrical impulses 150, also referred to as electrical stimulation, for transcutaneous electrical nerve stimulation (TENS). The controller 112 may be adjusted, that is, the controller 112 is adjustable, to control the intensity of the plurality of electrical impulses 150 produced by the control unit 110. The band 120 may be reversibly affixed to the control unit 110, and serves to securely, comfortable, and reversibly affix the apparatus 100 (that is, all of the apparatus 100 including the band 120) to an ankle 196, a foot 197, a leg 198, preferably the lower leg, or another body part of a patient 190. The plurality of electrical leads 130 are in electrical communication with the control unit 110 and with the plurality of electrode pads 140, such that the plurality of electrical impulses 150 may be transmitted from the control unit 110 through the plurality of electrical leads 130 to the plurality of electrode pads 140, where the plurality of electrical impulses 150 can penetrate a plurality of skin 192 of the patient 190, for the TENS therapy.

It has been found advantageous to treat patients with monosymptomatic NE, who are aged 5-18 years with approximately one month of behavioral therapy, followed by TENS therapy. The duration of the TENS therapy can be for approximately one month, or for up to approximately several months or approximately a year. In some cases, TENS therapy beyond a year may be used. With reference to FIG. 1, FIG. 2, FIG. 3, and FIG. 4, the plurality of electrode pads 140 have been used in a plurality of pad placement locations 142, including but not limited to a suprapubic pad location 144, a parasacral pad location 146, and an ankle posterior tibial pad location 148, which may also be referred to in the present disclosure as an ankle (posterior tibial) location. The ankle posterior tibial pad location 148 comprises a location for one or more of the plurality of electrode pads 140 on the ankle 196 and a location for one or more of the plurality of electrode pads 140 on the foot 197, referred to as a foot pad location 149. For simplicity in the present disclosure, the ankle posterior tibial pad location 148 is understood to include at least two from the plurality of electrode pads 140, one on the ankle 196 and one in the foot pad location 149. In a method 200 of TENS therapy treatment, TENS therapy, also referred to herein as TENS treatment, is performed nightly for approximately one month. A suprapubic pad location 144, for direct bladder stimulation, means the plurality of electrode pads 140 have a plurality of pad placement locations 142 on the abdomen directly over the bladder. A parasacral pad location 146, for an indirect proximal neural loop, means the plurality of electrode pads 140 have a plurality of pad placement locations 142 about 2-3 cm lateral to the midline in the sacral region at the level of the S3 vertebra. An ankle posterior tibial pad location 148, for an indirect distal neural loop, means the plurality of electrode pads 140 have a plurality of pad placement locations 142 on the bottom of a foot of the patient 190, and over a posterior portion of the medial malleolus on the ankle 196 that is ipsilateral thereto, of the patient 190.

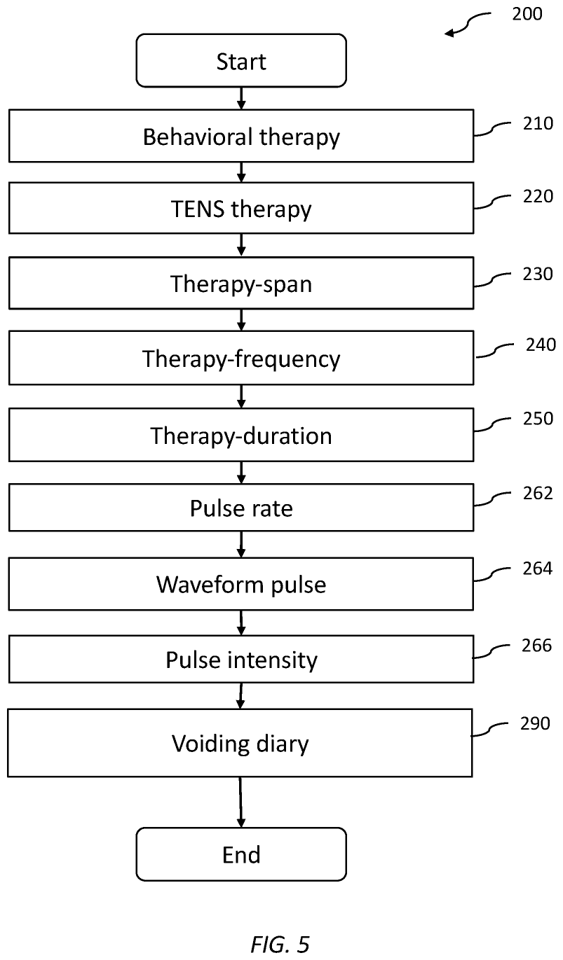

In development of the method 200, and with reference to FIG. 5, it has been found advantageous to have the patient 190 keep a voiding diary 290 recording the number of wet nights, wet scale severity score, which may be scored on a scale of 0-3 or other scale, TENS compliance—that is, compliance with the method 200 and protocols for use of the TENS therapy, wet accidents during the daytime, bowel movements to screen for constipation, and any adverse reactions. In the method 200, a pad placement at the ankle posterior tibial pad location 148 for the TENS therapy has shown significant improvements in all parameters, including significantly improved QOL during TENS therapy, along with robust improvements in the frequency of enuresis and severity of wetness each night after TENS therapy.

With reference to FIG. 5, the method 200 comprises a period of behavioral therapy 210, followed by TENS therapy 220, wherein the TENS therapy is conducted, preferably at home and by the patient 190 and/or a caregiver 194 of the patient 190, over a therapy-span 230, at a therapy-frequency 240, and each time the TENS therapy 220 is conducted, the TENS therapy 220 is conducted for a therapy-duration 250. In one aspect of the method 200, the therapy-span 230 is approximately a month, the therapy-frequency 240 is daily, and typically though not necessarily in the evening before bed, and the therapy-duration 250 is approximately 15 minutes.

The control unit 110 is notable for a component it omits, relatively to typical TENS units known in the art: the range of controls is greatly simplified. By omitting a control for waveform of the plurality of electrical impulses 150, the apparatus 100 of the present disclosure is significantly simplified, and made easier, safer, and more comfortable to use for children and their care-givers.

In one aspect of the present disclosure, the control unit 110 is set, for the TENS therapy 220 of the method 200, to a pulse rate 262 of approximately 10 Hz, normal mode, with a waveform pulse 264 that is, it has been found advantageous, rectangular with a width of approximately 270 us, therapy-duration 250 time set to 15 min, and pulse intensity 266 determined either programmatically, or by a medical provider based on the tolerance threshold of the patient 190, or by the patient 190 and/or the caregiver 194. It will be apparent to one of skill in the art that the foregoing pulse rates, waveforms, and waveform widths, and therapy-duration 250 times, as well as the intensity of the TENS therapy, are approximate values and others may be used. Without limiting the foregoing, the pulse rate 262 may be in or outside of a range of 0.1 Hz to 1,000 Hz; the waveform pulse 264 may have any shape or be variable; and/or the therapy-duration 250 may be in or outside of a range of 15 seconds to 2 hours.

The control unit 110 may further comprise an activation control 114, which may be a switch to control power on and power off of the apparatus 100, or may be a timer to activate the control unit 110 for a set or a user-programmable period of time. The control unit 110 may further comprise an intensity control 116, which may be a dial or knob or slider, or other mechanical or electrical component now known or later invented, to control the pulse intensity 266 of the TENS therapy 220. In some aspects of the present disclosure, the activation control 114 and the intensity control 116 may be integrated into a single mechanical or electrical component.

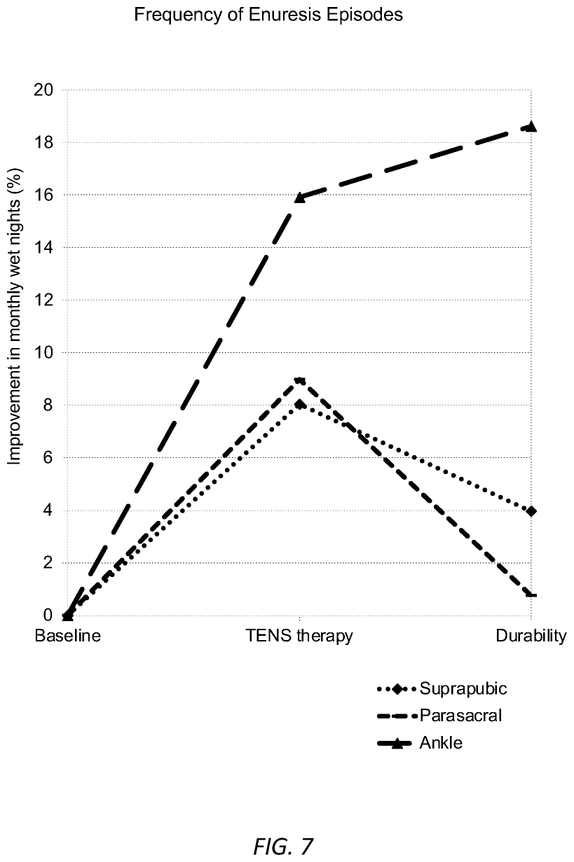

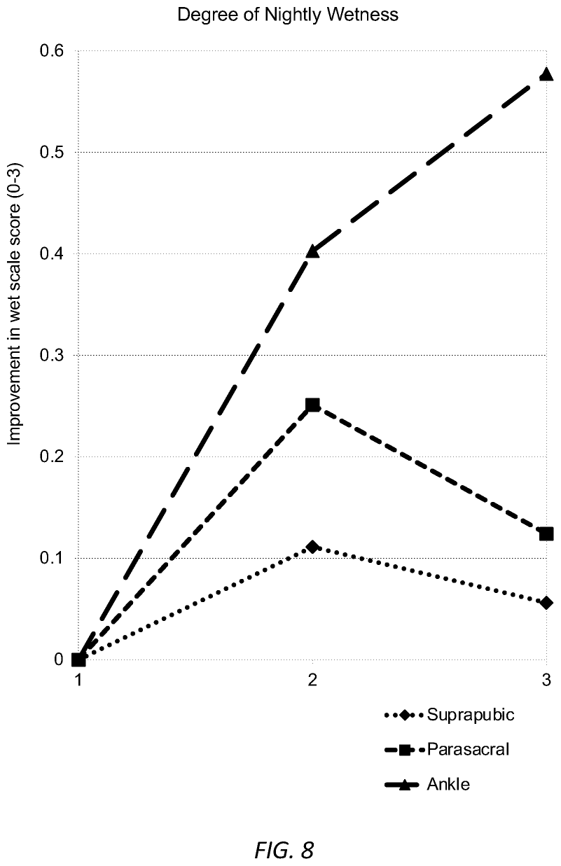

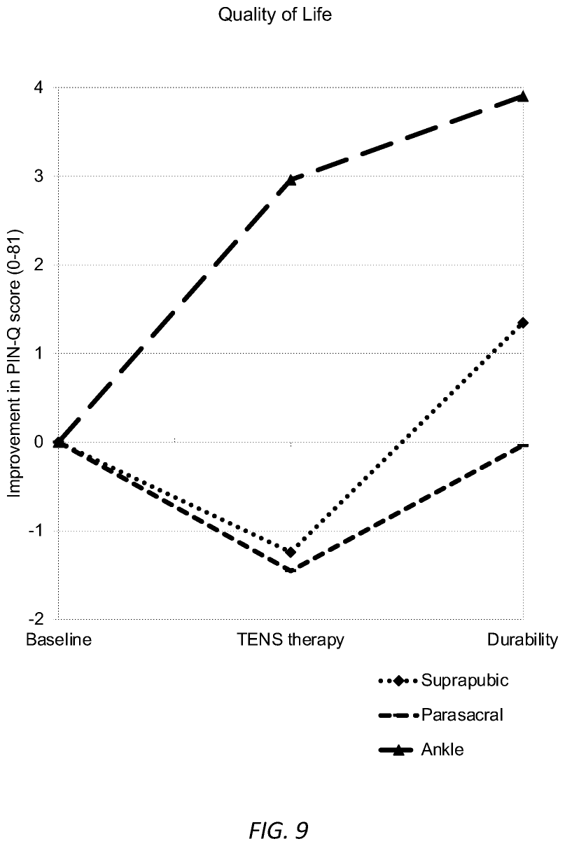

During treatment with the method 200 and apparatus 100, and with reference to FIG. 7 and FIG. 8, studied groups showed an improved response in both frequency and severity of enuresis from baseline, with the group using the plurality of pad placement locations 142 in the ankle posterior tibial pad location 148 showing the greatest results. Patient 190 QOL was improved during TENS therapy for only the ankle posterior tibial pad location 148 group, while patients in the groups with the plurality of pad placement locations 142 in the suprapubic pad location 144 and parasacral pad location 146 reported worsening of QOL, with reference to FIG. 9.

After treatment with the method 200 and apparatus 100, patients 190 showed the treatment effects to be durable, meaning the continued improvements in enuresis frequency, degree of nightly wetness, and QOL persisted for patients in the ankle posterior tibial pad location 148 group, showing the greatest sustainability in TENS effects each category. QOL did improved after TENS therapy was stopped in the suprapubic pad location 144 and in the parasacral pad location 146 groups, but the frequency and severity of enuresis deteriorated back towards the baseline, with reference to FIG. 7, FIG. 8, and FIG. 9. In studies conducted, for all patients 190 there was at least a partial response (2% full response, 4% good response, 18% partial response) to TENS therapy 220 following the method 200, as defined by the International Children's Continence Society (“ICCS”). The plurality of pad placement locations 142 at the ankle posterior tibial pad location 148 showed the greatest efficacy for patients 190 during the study in all data points, with significant improvement compared to others of the plurality of pad placement locations 142 with regards to PIN-Q scores (PIN-Q is a validated quality of life questionnaire used for children with voiding issues) during TENS therapy 220 using the method 200. With reference to Table 2, it is worth noting that the frequency of enuresis episodes and mean wet scale scores after TENS therapy in our durability assessment almost reached significance [p=0.06]. Additionally, the plurality of patients 190 in a study was asked about their satisfaction with the plurality of pad placement locations 142, and patient satisfaction was unanimous among the patients 190 in the ankle posterior tibial pad location 148 group, which was not the case with the patients 190 in the suprapubic pad location 144 groups and in the parasacral pad location 146 groups for the plurality of pad placement locations 142, with reference to Table 1, demonstrating another advantage of the apparatus 100 and method 200 of the present disclosure over the prior art.

TABLE 1
Demographics, patient compliance, and patient consideration of TENS.

| Group for | | | TENS | Would you | |
|---|---|---|---|---|---|
| pad | Mean age | Female, | compliance | use TENS again? | |
| placement | (range) | Male | % | Yes | No |
| Suprapubic | 11.5 (8-18) | 12, 18 | 89.6 | 16 | 2 |
| Parasacral | 10.4 (6-15) | 7, 23 | 92.5 | 9 | 2 |
| Ankle | 10.2 (6-17) | 9, 21 | 94.7 | 16 | 0 |
| P-value | 0.111* | | 0.131* | | 0.150** |

*Kruskal-Wallis test for non-normally distributed data
**Fisher's exact test

TABLE 2
Mean improvement changes in frequency of enuresis, degree of
wetness, and quality of life during and after TENS therapy.
Mean Improvement in Score (+/−Standard Deviation)

| | TENS Therapy | Durability |
|---|---|---|
| Frequency of Enuresis Episodes | | |
| Suprapubic | −8.04 (16.03) | 4.08 (21.56) |
| Parasacral | −8.98 (15.72) | 8.23 (17.66) |
| Ankle | −15.91 (21.10) | −2.71 (8.85) |
| P-value | 0.48 | 0.06 |
| Degree of Nightly Wetness | | |
| Suprapubic | 0.11 (0.78) | −0.06 (0.37) |
| Parasacral | 0.25 (0.58) | −0.13 (0.40) |
| Ankle | 0.40 (0.70) | 0.17 (0.20) |
| P-value | 0.48 | 0.06 |
| Quality of Life | | |
| Suprapubic | −1.2 (5.9) | 2.59 (6.1) |
| Parasacral | −1.5 (1.5) | 1.42 (6.1) |
| Ankle | 2.95 (5.3) | 0.94 (4.7) |
| P-value | 0.003 | 0.456 |

*P-values on Kruskal-Wallis test for non-normally distributed data

In a study conducted of the method 200, it was found that the ankle posterior tibial pad location 148 in the plurality of pad placement locations 142 for TENS therapy 220 had the greatest improvements in QOL, frequency and severity of NE in children with durability after treatment has commenced. The frequency and severity of nightly wetness after TENS therapy 220 ended showed net clinical benefits in the patients 190. At the end of the study, the patients 190 in the ankle posterior tibial pad location 148 group had an 18.6% decrease in enuresis frequency, which equates to 5.6 less wet nights per month and 1.4 wet nights per week—significant improvement in the quality of life for the patient 190 and the caregiver 194.

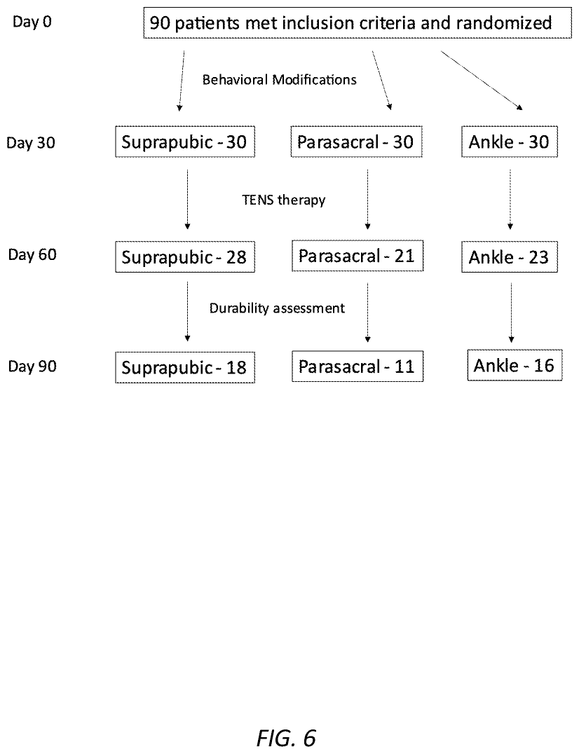

With reference to FIG. 6, a study was performed including a randomized clinical trial, measured multiple parameters showing improved QOL, frequency and severity of nocturnal enuresis, durability of treatment and improvements in patient outcomes, excellent compliance by patients, patient satisfaction, and safety with TENS therapy 220 at the parasacral pad location 146, without any adverse reactions or side effects. In the study, a total of 90 patients were randomized (Month 1), and 72 patients completed data for a full month of TENS therapy (Month 2), and 45 patients had follow-up data for durability assessment at 90 days (Month 3). The distribution of gender (mainly male) and age were similar in each group, as shown in Table 1. Behavioral modifications (Month 1) for all patients showed improved QOL with a mean improvement in PIN-Q score of 3.7 points and no significant differences between groups (p=0.97). Baseline data showed groups were similar in terms of QOL, NE frequency, and severity of wetness each night.

The study was also a head-to-head comparison of the plurality of pad placement locations 142 for TENS therapy 220 in treating nocturnal enuresis. The study conducted showed that the parasacral pad location 146 and the suprapubic pad location 144 for TENS therapy 220 were inferior locations and not helpful for treating nocturnal enuresis. The method 200 disclosed herein is developed as a protocol for a safe and easy-to-use home TENS therapy 220 that can be used before bed which is convenient to families, for the patient 190 and for the caregiver 194, and without medications.

Certain aspects of the present invention were described above. From the foregoing it will be seen that this invention is one well adapted to attain all the ends and objects set forth above, together with other advantages, which are obvious in and inherent to the inventive apparatus disclosed herein. It will be understood that certain features and sub-combinations are of utility and may be employed without reference to other features and sub-combinations. It is expressly noted that the present invention is not limited to those aspects described above, but rather the intention is that additions and modifications to what was expressly described herein are also included within the scope of the invention. Moreover, it is to be understood that the features of the various aspects described herein are not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. An apparatus for treatment of nocturnal enuresis, the apparatus comprising:
a control unit,
a controller, wherein the controller is in electrical or mechanical communication with the control unit,
a band,
a plurality of electrical leads, and
a plurality of electrode pads;
wherein the control unit generates a plurality of electrical impulses for transcutaneous electrical nerve stimulation (TENS).

2. The apparatus of claim 1, wherein the controller is adjustable to control an intensity of the plurality of electrical impulses.

3. The apparatus of claim 1, wherein the band is reversibly affixed to the control unit, and the band reversibly affixes the apparatus to an ankle, a foot, a leg, or another body part of a patient.

4. The apparatus of claim 1, wherein the plurality of electrical leads are in electrical communication with the control unit and with the plurality of electrode pads, such that the plurality of electrical impulses may be transmitted from the control unit through the electrical leads to the plurality of electrode pads, where the plurality of electrical impulses can penetrate a plurality of skin of a patient.

5. The apparatus of claim 1, wherein the plurality of electrode pads are used in a plurality of pad placement locations.

6. The apparatus of claim 1, wherein the plurality of electrode pads are used in an ankle (posterior tibial) pad location, and wherein the ankle (posterior tibial) pad location comprises a location for one or more of the plurality of electrode pads on an ankle and a location for one or more of the plurality of electrode pads on a foot, referred to as a foot pad location.

7. The apparatus of claim 6, wherein the ankle (posterior tibial) pad location, to create an indirect distal neural loop, means the plurality of electrode pads have a plurality of pad placement locations on a bottom of a foot of a patient, and over a posterior portion of a medial malleolus on the ankle that is ipsilateral thereto.

8. The apparatus of claim 1, wherein the control unit omits a control for waveform of the plurality of electrical impulses.

9. The apparatus of claim 1, wherein the control unit is set to a pulse rate of approximately 10 Hz, normal mode, with a waveform pulse that is rectangular with a width of approximately 270 us, a therapy-duration time set to 15 min, and a pulse intensity that is determined either programmatically, or by a medical provider based on a tolerance threshold of a patient, or by the patient and/or a caregiver.

10. The apparatus of claim 1, wherein the control unit further comprises an activation control and an intensity control.

11. A method for transcutaneous electrical nerve stimulation treatment (TENS treatment) of nocturnal enuresis, the method using an apparatus of claim 1, wherein the method comprises:
for a patient, reversibly affixing the apparatus to the patient, at least nightly; and
performing TENS therapy at a therapy-frequency for a therapy-span;
and wherein each time the TENS therapy is conducted, the TENS therapy is conducted for a therapy-duration.

12. The method of claim 11, wherein the method further comprises treating the patient with behavioral therapy for a duration of time prior to the TENS treatment.

13. The method of claim 12, wherein the duration of time prior to the TENS treatment is approximately one month.

14. The method of claim 11, wherein the method further comprises having the patient keep a voiding diary.

15. The method of claim 11, wherein the therapy-span is approximately a month.

16. The method of claim 11, wherein the therapy-frequency is daily.

17. The method of claim 11, wherein the therapy-duration is approximately 15 minutes.

18. The method of claim 11, wherein a control unit of the apparatus is set to a pulse rate of approximately 10 Hz, normal mode, with a waveform pulse that is rectangular with a width of approximately 270 us, a therapy-duration time set to 15 min, and a pulse intensity determined either programmatically, or by a medical provider based on a tolerance threshold of the patient, or by the patient and/or a caregiver.
